(19) United States
(12) Patent Application Publication
(10) Pub. No.: US 2008/0082061 A1
(43) Pub. Date: Apr. 3, 2008

(54) Draining Body Fluid from a Patient

(75) Inventor: Sui-Tao Zhou, Guangzhou (CN)

Correspondence Address: FISH & RICHARDSON, PC, P.O. BOX 1022, MINNEAPOLIS, MN, 55440-1022, US

(73) Assignee: Fei Pan

(21) Appl. No.: 11/864,221

(22) Filed: Sep. 28, 2007

(30) Foreign Application Priority Data
Sep. 28, 2006 (CN) 200610116637.2
Sep. 28, 2006 (CN) 200620046392.6
Mar. 1, 2007 (CN) 200710087607.8
Mar. 1, 2007 (CN) 200720004736.1

(51) Int. Cl. A61M 27/00

(52) U.S. Cl. 604/319

(57) ABSTRACT

Systems and techniques for draining body fluid from a patient. In one aspect, a device includes a body fluid inlet that is biasable to a negative pressure suitable for draining body fluid from a patient, a fine measurement chamber having a volume V1 and being connectable to the body fluid inlet to receive the body fluid and serve as an initial repository therefor, wherein the fine measurement chamber comprises a fine measurement mechanism having a resolution R1, and a coarse measurement chamber having a volume V2 and being connectable to and disconnectable from the fine measurement chamber via a valve to intermittently receive the body fluid and serve as a repository therefor, wherein the coarse measurement chamber comprises a coarse measurement mechanism having a resolution R2. Volume V1 is less than volume V2 and resolution R1 is greater than resolution R2.

### CROSS-REFERENCE TO RELATED APPLICATIONS

This application claims the priority of Chinese Patent Application Serial No. 200610116637.2, filed Sep. 28, 2006, Chinese Patent Application Serial No. 200620046392.6, filed Sep. 28, 2006, Chinese Patent Application Serial No. 200710087607.8, filed Mar. 1, 2007, and Chinese Patent Application Serial No. 200720004736.1, filed Mar. 1, 2007, the contents of all of which are incorporated herein by reference.

### BACKGROUND

This disclosure relates to systems and techniques for draining body fluid from a patient, especially from the pleural cavity and/or pericardial cavity.

One or more fluids can accumulate in a patient who is suffering from a medical condition. The accumulating fluids can include blood, lymph, and gas, as well as other fluids. The accumulation can result from acute events (such as trauma, chest surgery, or heart surgery) or chronic events (such as tuberculosis and pericarditis). The fluids can accumulate, e.g., in the pleural cavity and/or the pericardial cavity of a patient. A failure to drain such fluids can harm the patient and even lead to the death of the patient.

The rate at which fluids accumulate in the pleural cavity and/or pericardial cavity of a patient is often indicative of the physiological condition of the patient. For example, a rapid accumulation of blood in the pleural cavity and/or pericardial cavity of a patient can indicate that the patient is bleeding internally and that hemostatia may be warranted.

### SUMMARY

This disclosure describes systems and techniques for draining body fluid from the pleural cavity and/or pericardial cavity of a patient. In one aspect, a device includes a body fluid inlet that is biasable to a negative pressure suitable for draining body fluid from a patient, a fine measurement chamber having a volume V1 and being connectable to the body fluid inlet to receive the body fluid and serve as an initial repository therefor, wherein the fine measurement chamber comprises a fine measurement mechanism having a resolution R1, and a coarse measurement chamber having a volume V2 and being connectable to and disconnectable from the fine measurement chamber via a valve to intermittently receive the body fluid and serve as a repository therefor, wherein the coarse measurement chamber comprises a coarse measurement mechanism having a resolution R2. Volume V1 is less than volume V2 and resolution R1 is greater than resolution R2.

This and other aspects can include one or more of the following features. The device can include a pressure regulator arranged to maintain a pressure differential between the fine measurement chamber and the coarse measurement chamber. The coarse measurement chamber can be connectable to the fine measurement chamber to transfer the body fluid received by the fine measurement chamber under the pressure differential. The valve can be connected to the bottom of the fine measurement chamber.

The device can include a pressure balancing mechanism to compensate for a pressure change resulting from the transfer of the body fluid from the fine measurement chamber to the coarse measurement chamber. The device can include a manually operable member that is actuable to open the valve and transfer the body fluid from the fine measurement chamber to the coarse measurement chamber.

In another aspect, a method includes draining body fluid from one or both of the pleural cavity and the pericardial cavity of a patient, accumulating the drained body fluid in a fine measurement chamber that includes a fine measurement mechanism having a resolution R1, and transferring intermittently the body fluid accumulated in the fine measurement chamber to a coarse measurement chamber having a resolution R2. Transferring the body fluid comprises resetting the body fluid level in the fine measurement chamber to a reproducible baseline. Resolution R1 is greater than resolution R2.

This and other aspects can include one or more of the following features. The body fluid can be reset to the reproducible baseline by emptying the fine measurement chamber of the body fluid. Transferring the body fluid intermittently can include triggering the transfer of the body fluid and/or opening a valve separating the fine measurement chamber and the coarse measurement chamber. Transferring the body fluid intermittently can also include closing a valve on a fluid flow path between the fine measurement chamber and the patient. A pressure change resulting from the transfer of the body fluid from the fine measurement chamber to the coarse measurement chamber can be compensated for.

In another aspect, a method includes connecting a device for draining body fluid to one or both of the pleural cavity and the pericardial cavity of a patient, measuring a first amount of body fluid drained from the patient over a first time period using a fine measurement mechanism having a resolution R1, storing the first amount of body fluid in a chamber that includes a coarse measurement mechanism having a resolution R2, measuring a second amount of body fluid drained from the patient over a second time period using the fine measurement mechanism, storing the second amount of body fluid with the first amount of body fluid in the chamber that includes the coarse measurement mechanism, and measuring a third amount of body fluid drained from the patient over a third time period using the fine measurement mechanism. Resolution R1 is greater than resolution R2.

This and other aspects can include one or more of the following features. The first amount and the second amount can be stored by transferring the first amount and the second amount from a fine measurement chamber to the chamber. Transferring the first amount and the second amount can include resetting the body fluid level in the fine measurement chamber to a reproducible baseline and/or opening a valve to place the fine measurement chamber in communication with the chamber. Transferring the first amount and the second amount can also include transferring the first amount and the second amount by pressure-driving the first amount and the second amount.

Transferring the first amount and the second amount can include ending communication between fine measurement chamber and the one or both of the pleural cavity and the pericardial cavity of the patient.

These and other aspects can achieve one or more of the following advantages. Body fluid that is drained from the pleural cavity and/or pericardial cavity of a patient can initially be received in a fine measurement chamber. Relatively precise measurements of the amount of fluid drained from the patient can be made. Thereafter, the body fluid can be transferred by pressure-driving the body fluid from the fine measurement chamber into a larger coarse measurement chamber. For example, the body fluid can be transferred into the coarse measurement chamber after the passage of a convenient amount of time or as the fine measurement chamber begins to fill to capacity. Coarse measurements of the total amount of fluid drained from the pleural cavity and/or pericardial cavity of a patient can be made after transfer.

The details of one or more implementations are set forth in the accompanying drawings and the description below. Other features and advantages will be apparent from the description and drawings, and from the claims.

### DESCRIPTION OF DRAWINGS

FIG. 1 is a schematic representation of a system for draining fluid from a body.

FIG. 2 is a view from the rear of a device that can be used in the system of FIG. 1.

FIG. 3 is a view from above of the device of FIG. 2.

FIG. 4 is a view from the front of the device of FIG. 2.

FIG. 5 is an enlarged view of portions of the device of FIG. 2.

FIG. 6 is a diagrammatic representation of multi-path valve of the device of FIG. 2.

Like reference symbols in the various drawings indicate like elements.

### DETAILED DESCRIPTION

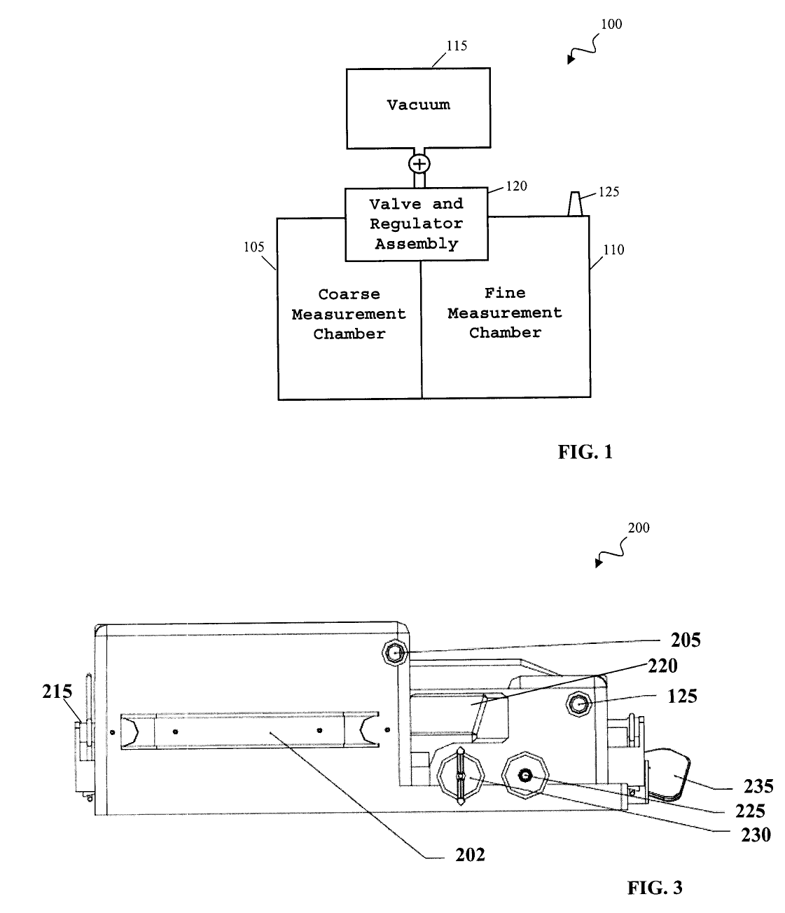

FIG. 1 is a schematic representation of a system 100 for draining fluid from a body. System 100 includes a coarse measurement chamber 105, a fine measurement chamber 110, a vacuum source 115, a valve and regulator assembly 120, and a fluid inlet 125. Coarse measurement chamber 105 is a fluid vessel that includes one or more mechanisms for making relatively coarse measurements of fluid amounts. Fine measurement chamber 110 is a fluid vessel that includes one or more mechanisms for making relatively fine measurements of fluid amounts. Vacuum source 115 is a source of a vacuum, such as a vacuum pump.

Valve and regulator assembly 120 is a collection of one or more valves and/or regulators that operatively interconnect coarse measurement chamber 105, fine measurement chamber 110, vacuum source 115 for draining fluid from a body over inlet 125. In particular, valve and regulator assembly 120 can regulate pressure transmitted from vacuum source 115 so that fine measurement chamber 110 can be kept at a negative pressure suitable for draining fluid from a body (e.g., between negative 20-25 cm H₂0) over fluid inlet 125. Valve and regulator assembly 120 can also valve the pressure differential between coarse measurement chamber 105 and fine measurement chamber 110 so that, at times, fluid is transferred by pressure-driving the fluid from fine measurement chamber 110 into coarse measurement chamber 105.

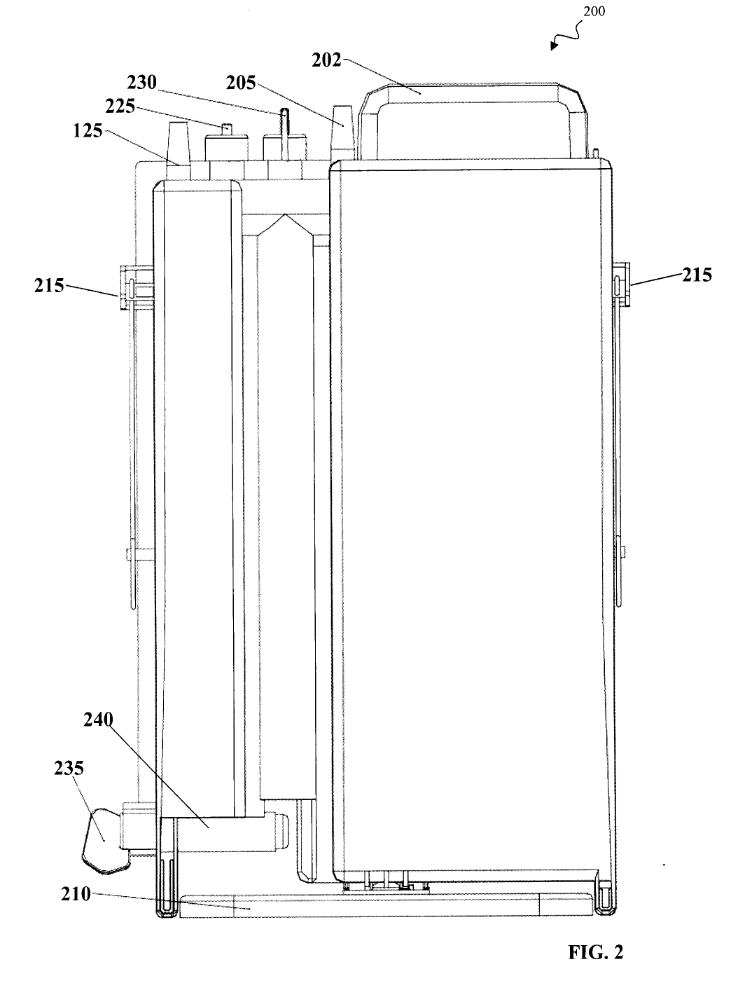

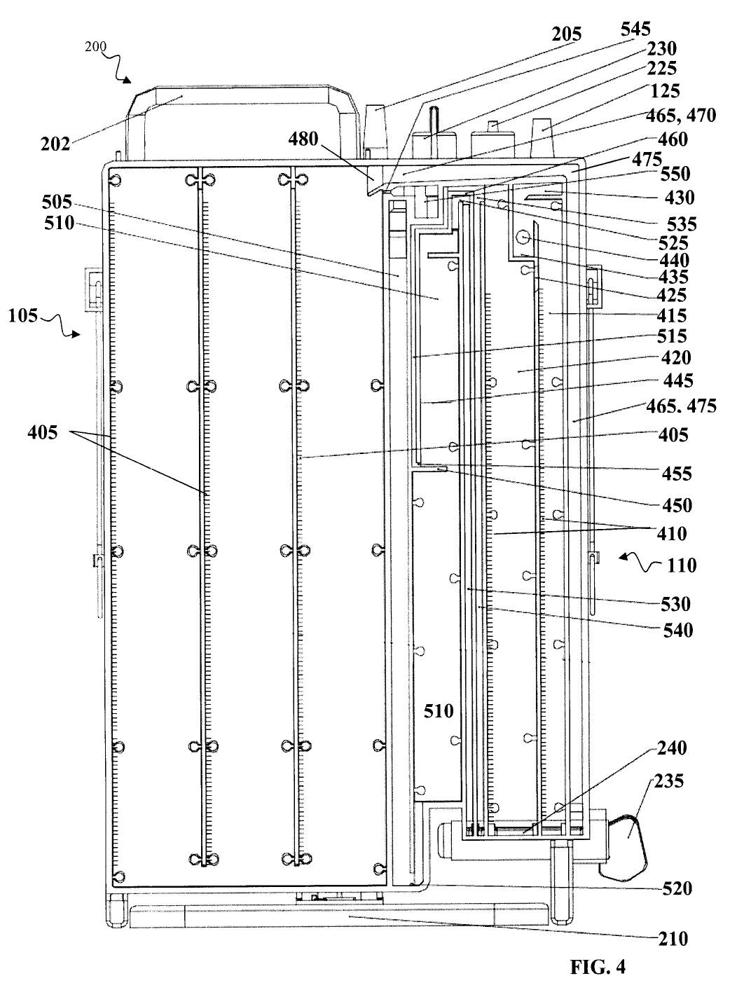

FIG. 2 is a view from the rear and FIG. 3 is a view from above of one implementation of a portion of system 100, namely, a device 200. Device 200 houses measurement chambers 105, 110 and valve and regulator assembly 120, as discussed further below. Device 200 can be connected to a hard or soft vacuum generated by vacuum source 115 over a vacuum inlet 205.

As shown, device 200 is a generally rectangular apparatus that is portable by medical personnel and/or a patient using a handle 202. Device 200 is mounted on a rotatable base 210 and can include one or more members for elevated mounting on a wall or a rack, such as a pair of hook assemblies 215. Using rotatable base 210 and/or hook assemblies 215, medical personnel can position device 200 appropriately relative to a patient.

Device 200 includes a vent opening 220 and a pair of valves 225, 230 that can place interior portions of device 200 in communication with the external environment. In particular, valve 225 is a pressure release valve that is connected to fine measurement chamber 110 within device 200. As discussed further below, pressure release valve 225 can act as an emergency pressure release and open to ensure that an excessive negative pressure is not applied to a patient over fluid inlet 125.

Valve 230 is an adjustable pressure regulator valve that is connected to a pressure regulator within device 200. As discussed further below, pressure regulator valve 230 can be adjusted to regulate the pressure in a chamber inside device 200 for use in draining body fluid from the pleural cavity and/or pericardial cavity of a patient.

Vent opening 220 vents a portion of the pressure regulator within device 200 that is connected to valve 230. Vent opening 220 can be covered with a removable air filtering membrane to allow nearly atmospheric pressure air to enter device 200 and ensure that regulation of pressure differential between coarse measurement chamber 105 and fine measurement chamber 110 is appropriate.

Device 200 also includes a valve knob 235. Valve knob 235 is manually-operable to open and close a multi-path valve 240 within device 200 and place coarse measurement chamber 105 and fine measurement chamber 110 in communication. By actuating valve knob 235, a human user can allow fluid collected in fine measurement chamber 110 to enter coarse measurement chamber 105.

FIG. 4 is a view from the front of device 200. The front of device 200 is formed of an at least partially transparent sheet that allows the fluid contents of measurement chambers 105, 110 inside device 200 to be visible from outside of device 200.

In this regard, coarse measurement chamber 105 is labeled with one or more collections of relatively coarse graduations 405 for volume measurement of the fluid contents of coarse measurement chamber 105. Fine measurement chamber 110 is labeled with one or more collections of relatively fine graduations 410 for volume measurement of the fluid contents of fine measurement chamber 110. A user looking through the transparent front sheet of device 200 can compare fluid levels in one or more of chambers 105, 110 to graduations 405, 410 to determine an amount of fluid that has been drained from the pleural cavity and/or pericardial cavity of a patient. Coarse measurement chamber 105 has a larger volume than fine measurement chamber 110 and can act as a relatively long-term reservoir for body fluid drained from the pleural cavity and/or pericardial cavity.

In the illustrated implementation, fine measurement chamber 110 is an assembly of measurement columns 415, 420 that are joined by a spill-over passage 425. Measurement column 415 is in communication with fluid inlet 125 via a guide passage 430 to receive fluid as it is drained from a patient. Spill-over passage 425 is positioned to allow fluid accumulating measurement column 415 to spill-over into measurement column 420. In some implementations, measurement column 420 can have the same diameter as measurement column 415 and can offer the same high (i.e., fine) resolution measurements of fluid volume. The arrangement of measurement columns 415, 420 and spill-over passage 425 allows a relatively large dynamic range of volumes to be measured at relatively high resolution.

Guide passage 430 is a fluid channel that guides fluid from fluid inlet 125 into measurement column 415, and hence into fine measurement chamber 110. In some implementations, guide passage 430 can include an upward facing concavity 435 that is accessible from outside of device 200 via an access port 440. Access port 440 can be a pressure-sealed port that allows a user to access fluid retained in upward facing concavity 435. In particular, as fluid is guided along guide passage 430 from fluid inlet 125 into measurement column 415, some portion of the fluid can be captured and retained in upward facing concavity 435. Access port allows a user to retrieve this portion of the fluid, e.g., for clinical or diagnostic purposes. In some implementations, access port 440 is a hole that is stoppered with a rubber plug.

Valve and regulator assembly 120 is also housed within device 200. Valve and regulator assembly 120 includes a vacuum feed system, a pressure regulator system, and a fluid transfer system. The vacuum feed system is a collection of passages within device 200 for feeding a vacuum placed on vacuum inlet 205 to various locations within device 200. The pressure regulator system regulates the pressure differential between coarse measurement chamber 105 and fine measurement chamber 110. The fluid transfer system allows fluid to be drained from fine measurement chamber 110 into coarse measurement chamber 105, as discussed further below.

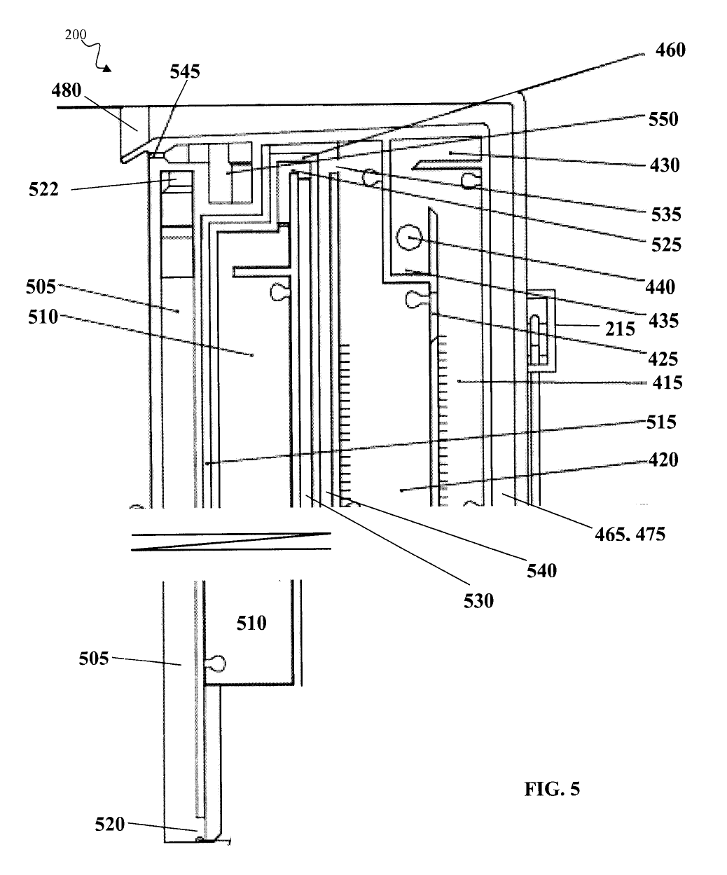

Portions of the vacuum feed system, the pressure regulator system, and the fluid transfer system of device 200 have enlarged in FIG. 5. As shown, the pressure regulator system includes a water infusion vent 505, a water storage column 510, and a pressure transmission channel 515. Infusion vent 505 extends from a top end 522 to an aperture 520. Top end 522 of water infusing vent 505 communicates with vent opening 220 and can be infused with liquid through vent opening 220. Aperture 520 of water infusing vent 505 communicates with the bottom water storage column 510.

Water storage column 510 extends from aperture 520 up to a connection 525 with a first pressure balance column 530. Pressure balance column 530 balances the pressure when fluid is transferred by pressure-driving the fluid from fine measurement chamber 110 into coarse measurement chamber 105, as discussed further below.

In some implementations, water storage column 510 can include a fill mark 445 and a baffle 450 along its height between aperture 520 and connection 525 of pressure balance column 530. Fill mark 445 identifies a height to which water storage column 510 can initially be loaded with liquid so that fluid can be drained from a body into device 200 at an appropriate negative pressure, as discussed further below. Baffle 450 is generally horizontal and demarcates upper and lower portions of water storage column 510. In particular, baffle 450 can aid medical personnel in distinguishing between venting gas that enters water storage column 510 by way of aperture 520 of water infusing vent 505 and gas drained from a patient that enters water storage column 510 by way of pressure transmission channel 515. Fill mark 445 is generally positioned along water storage column 510 above baffle 450.

Pressure transmission channel 515 runs alongside a portion of water storage column 510 and extends between an upper inlet 460 and an outlet 455. Upper inlet 460 places pressure transmission channel 515 in communication with an extent 535 of fine measurement chamber 110. Outlet 455 is positioned above baffle 450 and places channel 515 in communication with water storage column 510. Thus, channel 515 places fine measurement chamber 110 in communication with water storage column 510 for the transmission of a regulated pressure from water storage column 510 to fine measurement chamber 110, as discussed further below.

As discussed above, the vacuum feed system is a collection of passages for feeding a vacuum placed on vacuum inlet 205 to various locations within device 200. In particular, the vacuum feed system includes a first vacuum feed 545 and a second vacuum feed 550. Vacuum feed 545 is arranged to feed a vacuum placed on vacuum inlet 205 to a junction between pressure regulator valve 230 and water storage column 510. The negative pressure at vacuum inlet 205 is decreased by first vacuum feed 545, regulated by pressure regulator valve 230, and transferred by second vacuum feed 550 to water storage column 510. Vacuum feed 550 is arranged to feed a vacuum placed on vacuum inlet 205 to a junction between pressure regulator valve 230 and water storage column 510. Pressure regulator valve 230 can regulates the pressure transferred by vacuum feed 550 to water storage column 510 so that water storage column 510 is maintained at a pressure that is suitable for draining body fluid from the pleural cavity and/or pericardial cavity, as discussed further below.

As discussed above, the fluid transfer system allows fluid to be transferred from fine measurement chamber 110 into coarse measurement chamber 105. In addition to multi-path valve 240, the fluid transfer system includes a fluid transfer path 465 and a pair of pressure balance columns 530, 540.

Fluid transfer path 465 include a downwardly sloped component 470 and a generally vertical component 475. Generally vertical component 475 extends from multi-path valve 240, which is located at the base of fine measurement chamber 110, upward to communicate with downwardly sloped component 470 at a position 475. Downwardly sloped component 470 slopes downward from position 475 to enter coarse measurement chamber 105 at an inlet 480.

Pressure balance column 530 extends between connection 525 with water storage column 510 and multi-path valve 240. Pressure balance column 540 between multi-path valve 240 a connection 535 with fine measurement chamber 110. Multi-path valve 240 is operable to open and close connection between pressure balance columns 530, 540.

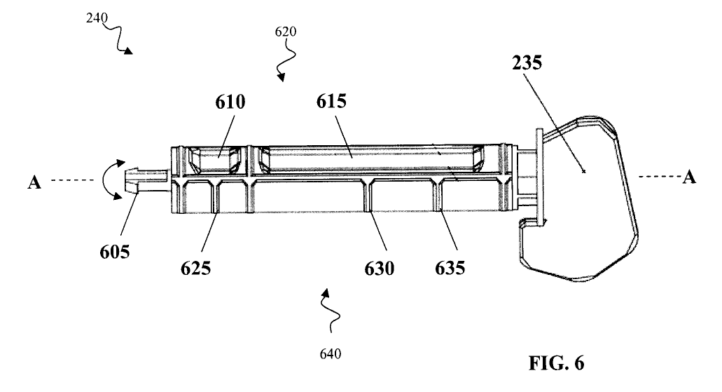

FIG. 6 is a diagrammatic representation of multi-path valve 240. As discussed above, multi-path valve 240 can be actuated using valve knob 235. Multi-path valve 240 is generally tubular in shape and extends longitudinally from valve knob 235 to a pivot 605. A pair of longitudinal paths 610, 615 are defined adjacent to one another on a first side 620 of multi-path valve 240. A collection of ridges 625, 630, 635 are oriented radially on a second side 640 of multi-path valve 240.

Pivot 605 can be compression fit within a hole on device 200 to position multi-path valve 240 in device 200 but yet allow rotation about an axis A. In particular, when properly positioned, multi-path valve 240 can be rotated to switch between an open and a closed position. In the open position, pressure balance columns 530, 540 are in communication via longitudinal path 610 and measurement columns 415, 420 are in communication with each other and with generally vertical component 475 of fluid transfer path 465 via longitudinal path 615. In the closed position, communication between pressure balance columns 530, 540 is prevented by ridge 625, communication between measurement columns 415, 420 is prevented by ridge 630, and communication between measurement column 420 and generally vertical component 475 of fluid transfer path 465 is prevented by ridge 635. In some implementations, communication can be prevented by the mating of ridges 625, 630, 635 with a polymeric or other sealing member.

In operation, device 200 can be positioned relative to a patient and supported using base 210 and/or hook assemblies 215. Water, saline solution, or other liquid can be poured into device 200 through vent opening 220. This liquid will enter water infusing vent 505 and, via aperture 520, water storage column 510. When the water reaches fill mark 445 of water storage column 510, the filling of device 200 can be halted. Vent opening 220 can then be covered with an air filtering membrane.

A vacuum source 115 place a vacuum on vacuum inlet 205 of device 200 using, e.g., a valved tube that is initially closed. An end of a sterile chest tube that is connected to an initially closed valve can be placed in the pleural cavity and/or pericardial cavity of a patient. The other side of the valve can be connected to fluid inlet 125. If necessary, pressure regulator valve 230 can be adjusted so that the pressure in fine measurement chamber 110 is appropriate for drawing body fluid from the pleural cavity and/or pericardial cavity. For example, the approximately pressure regulator valve 230 can be adjusted to provide a negative pressure of about 20 to 25 cm H₂O in fine measurement chamber 110. The vacuum provided by vacuum source 115 can be released into device 200.

The vacuum provided by vacuum source 115 is fed to coarse measurement chamber 105 via vacuum inlet 205 and to water storage column 510 via vacuum feed 545, pressure regulator valve 230, and vacuum feed 550. The application of the vacuum regulated by pressure regulator valve 230 to water storage column 510 will cause the water level in water storage column 510 to rise above fill mark 445, draining water infusing vent 505. At some point, the water level in water infusing vent 505 will fall below the level of aperture 520 and atmospheric air will pass into water storage column 510 via vent opening 220 and water infusing vent 505. This air will be visible to a user as bubbles that indicate to a user that the pressure in water storage column 510 is appropriate. In particular, water infusing vent 505, water storage column 510, and aperture 520 can be positioned and dimensioned so that a column of about 20-25 cm is maintained in water storage column 510, or about 2-3 cm above outlet 455.

At this point, the initially closed valve on the chest tube can be opened. The regulated negative pressure in water storage column 510 is transmitted to extent 535 of fine measurement chamber 110 via pressure transmission channel 515. Since measurement columns 415, 420 are in communication via spill-over passage 425, the regulated negative pressure is transmitted to guide passage 430, fluid inlet 125, and ultimately to the pleural cavity and/or pericardial cavity of the patient.

This regulated negative pressure will drain body fluid from the pleural cavity and/or pericardial cavity of the patient through fluid inlet 125 and guide passage 430 and into fine measurement chamber 110 where it is initially collected in measurement column 415. With valve 240 in the closed position, body fluid accumulates in measurement column 415. The amount of body fluid accumulated in measurement column 415 can be determined by comparing the body fluid level with relatively fine graduations 410. At some point, the body fluid accumulated in measurement column 415 will rise to the level of spill-over passage 425. Spill-over passage 425 allows the body fluid to spill over into measurement column 420. The amount of body fluid accumulated in measurement column 420 can also be determined using relatively fine graduations 410.

In some implementations, medical personnel can sample the body fluid accumulating in fine measurement chamber 110 via access port 440. For example, when access port 440 is a hole that is stoppered with a rubber plug, medical personnel can use a syringe or other needle to penetrate the plug and withdraw fluid.

At some point, the fluid accumulating in fine measurement chamber 110 can be transferred to coarse measurement chamber 105. This may be triggered, e.g., by the fluid accumulation in fine measurement chamber 110 approaching the capacity of fine measurement chamber 110 or the passage of a physiologically suitable measurement time.

To transfer the accumulated body fluid from fine measurement chamber 110 to coarse measurement chamber 105, the valve on the chest tube can be closed. Valve knob 235 can be actuated to rotate multi-path valve 240 from the closed position to the open position. As discussed above, in the open position, measurement columns 415, 420 are in communication with each other and with generally vertical component 475 of fluid transfer path 465 via longitudinal path 615. Fluid transfer path 465 communicates with coarse measurement chamber 105 which is under vacuum via inlet 205. Thus, the accumulated body fluid will be drawn from fine measurement chamber 110, up generally vertical component 475 and down downwardly sloped component 470 of fluid transfer path 465 into coarse measurement chamber 105.

Moreover, as discussed above, rotation of multi-path valve 240 into the open position also places pressure balance columns 530, 540 in communication with each other via longitudinal path 610. The transfer of fluid from fine measurement chamber 110 into coarse measurement chamber 105 draws air into pressure balance columns 530, 540 via water storage column 510, water infusing vent 505, and vent opening 220. This allows fine measurement chamber 110 to be rapidly emptied of fluid so that subsequent measurements of fluid accumulation in fine measurement chamber 110 can begin from an easily reproducible baseline.

Once fine measurement chamber 110 has been drained, valve knob 235 can be actuated to rotate multi-path valve 240 from the open position to the closed position. Communication between pressure balance columns 530, 540 cut off, as is communication between measurement columns 415, 420, and generally vertical component 475 of fluid transfer path 465. Moreover, any residual liquid in downwardly sloped component 470 of fluid transfer path 465 can drain, via gravity, into coarse measurement chamber 105 and the regulated negative pressure in water storage column 510 will be restored.

At this point, the valve on the chest tube can be reopened and body fluid can again be drained from the pleural cavity and/or pericardial cavity of the patient through fluid inlet 125 and guide passage 430 and into fine measurement chamber 110.

In some implementations, graduations 410 of fine measurement chamber 110 can allow a user to measure volumes up to 100-200 ml with a resolution of ±5 ml and graduations 405 of coarse measurement chamber 105 can allow a user to measure volume up to 1000 ml-2000 ml with a resolution of ±10 ml-20 ml.

A number of implementations have been described. Nevertheless, it will be understood that various modifications may be made. For example, valve knob 235 is shown as a manually operated knob that actuates multi-path valve 240. However, multi-path valve 240 can also be operated automatically and/or electronically. For example, device 200 can include a level sensor that triggers the opening and closing of multi-path valve 240. As another example, fine measurement chamber 110 can be assembled from additional measurement columns that are joined by additional spill-over passages.

As another example, the pressure regulator system can include a dye or other marker to color the liquid in water infusing vent 505 and water storage column 510 for easy identification by a user.

As another example, other measurement mechanisms, including electronic and/or mass measurement mechanisms, can be used in coarse measurement chamber 105 and/or fine measurement chamber 110.

Accordingly, other implementations are within the scope of the following claims.

What is claimed is:

1. A device comprising:
a body fluid inlet that is biasable to a negative pressure suitable for draining body fluid from a patient;
a fine measurement chamber having a volume V1 and being connectable to the body fluid inlet to receive the body fluid and serve as an initial repository therefor, wherein the fine measurement chamber comprises a fine measurement mechanism having a resolution R1; and
a coarse measurement chamber having a volume V2 and being connectable to and disconnectable from the fine measurement chamber via a valve to intermittently receive the body fluid and serve as a repository therefor, wherein the coarse measurement chamber comprises a coarse measurement mechanism having a resolution R2,
wherein volume V1<volume V2 and
resolution R1>resolution R2.

2. The device of claim 1, further comprising a pressure regulator arranged to maintain a pressure differential between the fine measurement chamber and the coarse measurement chamber.

3. The device of claim 2, wherein the coarse measurement chamber is connectable to the fine measurement chamber to transfer the body fluid received by the fine measurement chamber under the pressure differential.

4. The device of claim 1, wherein the valve is connected to the bottom of the fine measurement chamber.

5. The device of claim 1, further comprising a pressure balancing mechanism to compensate for a pressure change resulting from the transfer of the body fluid from the fine measurement chamber to the coarse measurement chamber.

6. The device of claim 1, further comprising a manually operable member that is actuable to open the valve and transfer the body fluid from the fine measurement chamber to the coarse measurement chamber.

7. A method comprising:
draining body fluid from one or both of the pleural cavity and the pericardial cavity of a patient;
accumulating the drained body fluid in a fine measurement chamber that includes
a fine measurement mechanism having a resolution R1; and
transferring intermittently the body fluid accumulated in the fine measurement chamber to a coarse measurement chamber having a resolution R2,
wherein transferring the body fluid comprises resetting the body fluid level in the fine measurement chamber to a reproducible baseline, and
resolution R1>resolution R2.

8. The method of claim 7, wherein resetting the body fluid to the reproducible baseline comprises emptying the fine measurement chamber of the body fluid.

9. The method of claim 7, wherein transferring the body fluid intermittently comprises triggering the transfer of the body fluid.

10. The method of claim 7, wherein transferring the body fluid intermittently comprises opening a valve separating the fine measurement chamber and the coarse measurement chamber.

11. The method of claim 7, wherein transferring the body fluid intermittently comprises closing a valve on a fluid flow path between the fine measurement chamber and the patient.

12. The method of claim 7, further comprising compensating for a pressure change resulting from the transfer of the body fluid from the fine measurement chamber to the coarse measurement chamber.

13. A method comprising:
connecting a device for draining body fluid to one or both of the pleural cavity and the pericardial cavity of a patient;
measuring a first amount of body fluid drained from the patient over a first time period using a fine measurement mechanism having a resolution R1;
storing the first amount of body fluid in a coarse measurement chamber that includes a coarse measurement mechanism having a resolution R2;
measuring a second amount of body fluid drained from the patient over a second time period using the fine measurement mechanism;
storing the second amount of body fluid with the first amount of body fluid in the coarse measurement chamber that includes the coarse measurement mechanism; and
measuring a third amount of body fluid drained from the patient over a third time period using the fine measurement mechanism,
wherein R1>R2.

14. The method of claim 13, wherein storing the first amount and storing the second amount comprises transferring the first amount and the second amount from a fine measurement chamber to the coarse measurement chamber.

15. The method of claim 14, wherein transferring the first amount and the second amount comprises resetting the body fluid level in the fine measurement chamber to a reproducible baseline.

16. The method of claim 14, wherein transferring the first amount and the second amount comprises opening a valve to place the fine measurement chamber in communication with the coarse measurement chamber.

17. The method of claim 14, wherein transferring the first amount and the second amount comprises transferring the first amount and the second amount by pressure-driving the first amount and the second amount.

18. The method of claim 14, wherein transferring the first amount and the second amount comprises ending communication between a fine measurement chamber and the one or both of the pleural cavity and the pericardial cavity of the patient.
